(19) United States
(12) Patent Application Publication
(10) Pub. No.: US 2011/0027881 A1
(43) Pub. Date: Feb. 3, 2011

(54) PRODUCTION METHOD OF IMMUNE CELLS

(75) Inventors: Ken-ichiro SEINO, Kawasaki-shi (JP); Haruka WADA, Kawasaki-shi (JP)

Correspondence Address: THE NATH LAW GROUP, 112 South West Street, Alexandria, VA, 22314, US

(73) Assignee: St. Marianna University School of Medicine, Kawasaki-shi (JP)

(21) Appl. No.: 12/845,178

(22) Filed: Jul. 28, 2010

Related U.S. Application Data
(60) Provisional application No. 61/213,940, filed on Jul. 31, 2009.

(51) Int. Cl. C12N 5/00

(52) U.S. Cl. 435/373

(57) ABSTRACT

A production method of T cells is disclosed which includes generating iPS cells from immune cells and differentiating the iPS cells into desired immune cells. In this method, 4 different genes Oct4, Sox2, Klf4 and c-Myc are introduced into immune cells for generation of iPS cells, and the iPS cells are then differentiated into immune cells by coculture with OP9 cells. Source immune cells are taken from a patient, and the produced desired immune cells are injected into the patient for medical treatment.

### CROSS REFERENCE TO RELATED APPLICATIONS

This application is entitled to the benefit of U.S. Provisional Patent Application No. 61/213,940, filed on Jul. 31, 2009, the disclosure of which is incorporated herein by reference in its entirety.

### TECHNICAL FIELD

This disclosure relates to production methods of immune cells such as lymphocytes.

2. Background Art

Pluripotency can be induced in human and mouse somatic cells by the forced expression of OCT4 (Oct4) and SOX2 (Sox2) with either the combinations of KLF4 (Klf4) and c-MYC (c-Myc) or NANOG (Nanog) and LIN28 (Lin28) (see Non Patent Literatures 1-4). Differentiation of induced pluripotent stem (iPS) cells into various cells belonging to the three germ layers has been demonstrated by the analysis of teratomas generated from human and mouse iPS cells. In addition, the pluripotency of iPS cells is obvious by the contribution of iPS cell-derived cells to various organs of the chimeric mice developed from iPS cell-introduced blastocysts (see Non Patent Literature 5).

Recently, derivation of mouse iPS cell lines from bone marrow hematopoietic progenitor cells has been reported (see Non Patent Literature 6). Derivation of iPS cells from postnatal human blood cells has been also reported. Loh et al. reported derivation of iPS cells from granulocyte colony-stimulating factor (G-CSF) mobilized peripheral blood CD34⁺ cells (see Non Patent Literature 7). Further, Ye et al. reported derivation of iPS cells from human cord blood and adult bone marrow CD34⁺ cells without any pre-treatment such as G-CSF mobilization (see Non Patent Literature 8). These reports all employed hematopoietic progenitor or stem cells as the source of iPS cells.

It has also been reported that T cells are used as the source of iPS cells (see Non Patent Literatures 9-11). Hanna et al. reported derivation of iPS cells from murine B cells (see Non Patent Literature 9). In this report, it was indicated that only pro- and pre-B cells could be reprogrammed with 4 reprogramming factors—Oct4, Sox2, KlF and c-Myc—whereas mature B cells could be reprogrammed by the additional overexpression of C/EBPα or specific knockdown of the Pax5 transcription factor. Eminli et al. also reported that iPS cells were established from terminally differentiated B and T cells by overexpression of the 4 factors, although the efficiency was quite low compared to hematopoietic stem and progenitor cells (see Non Patent Literature 10). In these studies, iPS cells were derived from primary B or T cells of mice engineered to carry doxycycline-inducible Oct4, Sox2, Klf4 and c-Myc retroviruses in every tissue (see Non Patent Literatures 9 and 10). Similarly, Hong et al. recently reported that murine splenic T cells of p53-null mice could be reprogrammed to iPS cells (see Non Patent Literature 11). These studies suggest that it is difficult to establish iPS cells from mature B or T cells using only the so-called Yamanaka 4 factors (Oct4, Sox2, KlF and c-Myc) unless additional modification is given.

As for the in vitro generation of cells of mesodermal lineage from iPS cells, differentiation into cardiac myocytes and endothelial cells from iPS cells has been recently reported (see Non Patent Literatures 12-14). Senju et al. recently reported that mouse iPS cells can differentiate into macrophages and dendritic cells (see Non Patent Literature 15). Lei et al. recently reported that mouse iPS cells can differentiate into T cells by coculture with OP9-DL1 cells (see Non Patent Literature 16).

Schmitt et al. have indicated that B cells can be differentiated by day 20 from embryonic or hematopoietic stem cells cultured on OP9 cells in the presence of F1t3L and IL-7 (see Non Patent Literature 17).

### CITATION LIST

### Non Patent Literature

NPL1: Park I H, Zhao R, West J A et al., “Reprogramming of human somatic cells to pluripotency with defined factors”, Nature, Vol. 451, pp. 141-146.

NPL2: Takahashi K, Tanabe K, Ohnuki M et al., “Induction of pluripotent stem cells from adult human fibroblasts by defined factors”, Cell, Vol. 131, pp. 861-872.

NPL3: Takahashi K, Yamanaka S., “Induction of pluripotent stem cells from mouse embryonic and adult fibroblast cultures by defined factors”, Cell, Vol. 126, pp. 663-676.

NPL4: Yu J, Hu K, Smuga-Otto K et al., “Human induced pluripotent stem cells free of vector and transgene sequences”, Science, Vol. 324, pp. 797-801.

NPL5: Okita K, Ichisaka T, Yamanaka S., “Generation of germline-competent induced pluripotent stem cells”, Nature, Vol. 448, pp. 313-317.

NPL6: Okabe M, Otsu M, Ahn D H et al., “Definitive proof for direct reprogramming of hematopoietic cells to pluripotency”, Blood, Vol. 114, pp. 1764-1767.

NPL7: Loh Y H, Agarwal S, Park IH et al., “Generation of induced pluripotent stem cells from human blood”, Blood, Vol. 113, pp. 5476-5479.

NPL8: Ye Z, Zhan H, Mali P et al., “Human induced pluripotent stem cells from blood cells of healthy donors and patients with acquired blood disorders”, Blood, Vol. 114, pp. 5473-5480.

NPL9: Hanna J, Markoulaki S, Schorderet P et al., “Direct reprogramming of terminally differentiated mature B cells to pluripotency”, Cell, Vol. 133, pp. 250-264.

NPL10: Eminli S, Foudi A, Stadtfeld M et al., “Differentiation stage determines potential of hematopoietic cells for reprogramming into induced pluripotent stem cells”, Nat. Genet., Vol. 41, pp. 968-976.

NPL11: Hong H, Takahashi K, Ichisaka T et al., “Suppression of induced pluripotent stem cell generation by the p53-p21 pathway”, Nature, Vol. 460, pp. 1132-1135.

NPL12: Mauritz C, Schwanke K, Reppel M et al., “Generation of functional murine cardiac myocytes from induced pluripotent stem cells”, Circulation, Vol. 118, pp. 507-517.

NPL13: Narazaki G, Uosaki H, Teranishi M et al., “Directed and systematic differentiation of cardiovascular cells from mouse induced pluripotent stem cells”, Circulation, Vol. 118, pp. 498-506.

NPL14: Schenke-Layland K, Rhodes K E, Angelis E et al., “Reprogrammed mouse fibroblasts differentiate into cells of the cardiovascular and hematopoietic lineages”, Stem Cells, Vol. 26, pp. 1537-1546.

NPL15: Senju S, Haruta M, Matsunaga Y et al., “Characterization of dendritic cells and macrophages generated by directed differentiation from mouse induced pluripotent stem cells”, Stem Cells, Vol. 27, pp. 1021-1031.

NPL16: Lei F, Hague R, Weiler L et al., “T lineage differentiation from induced pluripotent stem cells”, Cell Immunol., Vol. 260, pp. 1-5.

NPL17: Schmitt T M, de Pooter R F, Gronski M A et al., “Induction of T cell development and establishment of T cell competence from embryonic stem cells differentiated in vitro”, Nat. Immunol., Vol. 5, pp. 410-417.

### SUMMARY OF INVENTION

### Technical Problem

It is highly attractive to utilize recently developed iPS cell production techniques for the development of novel immunotherapy against cancer or infection. It has been common in the art to employ adherent cells such as fibroblasts and keratinocytes as the source of iPS cells. However, preparation of patient-specific iPS cells from adherent cells requires skin biopsies and subsequent subculture, imposing a significant burden on the patient and making the procedure cumbersome. These disadvantages can be overcome if patient-specific iPS cells can be produced from peripheral blood immune cells; however, as described above, it has been difficult to induce iPS cells from peripheral blood mature immune cells. Moreover, in vitro differentiation of iPS cells into immune cells (particularly T cells) has also been difficult.

It is therefore an object of the present invention to provide a production method of immune cells which includes the steps of generating iPS cells from immune cells, which can be readily collected from the patient's body, and differentiating the iPS cells into desired immune cells.

### Solution to Problem

The inventors established that, by introducing reprogramming factors two or more times into immune cells, iPS cells can be generated from the immune cells with only 4 reprogramming factors, and that the iPS cells can be differentiated into immune cells by coculuture with OP9 cells. The inventors thus conducted additional studies to accomplish the present invention.

Specifically, the following production methods of immune cells are provided.

[1] A production method of immune cells including:

generating induced pluripotent stem cells from source immune cells; and

differentiating the induced pluripotent stem cells into immune cells by coculture with OP9 cells.

[2] The method according to [1], wherein the source immune cells are mature B cells, and the induced pluripotent stem cells are produced by introducing only reprogramming factors OCT4, SOX2, KLF4 and c-MYC into the mature B cells.

[3] The method according to [2], wherein the induced pluripotent stem cells are produced by introducing the reprogramming factors two or more times into the mature B cells.

[4] The method according to any one of [1] to [3], wherein the OP9 cells are OP9-DL1 cells expressing Notch ligand delta like 1, and the induced pluripotent stem cells are differentiated into T cells by coculture with the OP9-DL1 cells.

### ADVANTAGEOUS EFFECTS OF INVENTION

The present invention enables semipermanent bulk production of desired immune cells from source immune cells which are readily collected from the patient's body. The produced immune cells can then be used for immunotherapy.

### BRIEF DESCRIPTION OF DRAWINGS

FIG. 1 is a picture of iPS cells derived from mouse CD19⁺ B cells (B-iPS cells);

FIG. 2 is a gel electrophoresis picture indicating the expression of ES cell marker genes in B-iPS cells;

FIG. 3 is a gel electrophoresis picture indicating the occurrence of IgH V(D)J rearrangements in B-iPS cells;

FIG. 4A is a picture of ciliated cells in teratoma;

FIG. 4B is a picture of muscle fiber in teratoma;

FIG. 4C is a picture of dermal tissue in teratoma;

FIG. 5 is a picture of a chimeric mouse produced using B-iPS cells;

FIG. 6A is a picture of spherical bodies derived from B-iPS cells at day 5 of culture;

FIG. 6B shows histograms indicating the patterns of Flk-1 expression in cells constituting spherical bodies derived from iPS cells (B-iPS cells or MEF-iPS cells);

FIG. 6C is a picture of lymphocyte-like cells derived from B-iPS cells;

FIG. 7 shows dot plots indicating the patterns of differentiation marker expression in cells derived from iPS cells (B-iPS cells or MEF-iPS cells);

FIG. 8 shows histograms indicating the patterns of Tcrβ gene expression in T cells derived from B-iPS cells;

FIG. 9 is a gel electrophoresis picture showing the results of genomic polymerase chain reaction (PCR) for B-iPS cells, differentiated cells from B-iPS cells, OP9-DL1 cells, and mouse adult thymocytes;

FIG. 10 shows a dot plot indicating the patterns of TCRβ and TCRγδ expressions;

FIG. 11 shows dot plots indicating that iPS cell-derived T cells produce IFN-γ;

FIG. 12 shows dot plots indicating that iPS cell-derived T cells express PoxP3 in response to TGF-β1; and

FIG. 13 is a gel electrophoresis picture showing the results of Reverse transcription (RT)-PCR for iPS cells (B-iPS cells or MEF-iPS cells) cocultured with OP9-DL1 and for thymocytes.

### DESCRIPTION OF EMBODIMENTS

The production method of immunocytes includes (1) a first step of generating iPS cells from source immunocytes, and (2) a second step of differentiating the iPS cells into immune cells by coculture with OP9 cells.

In the first step, iPS cells are generated from source immune cells.

There are no particular limitations to the source immune cells; any desired immune cells can be employed. When the immune cells produced by the production method according to an embodiment are intended to be used for immunotherapy, source immune cells are selected from human immune cells. The source immune cells may be either fetal or adult immune cells, and may be either immature or mature immune cells. Examples of source immune cells include T cells, B cells, NK cells, NKT cells, dendritic cells, moncytes, and granulocytes.

There are no particular limitations to the method of establishing iPS cells from immune cells; it can be selected from any known method. For example, retroviral transduction may be employed to introduce the 4 genes OCT4 (Oct4), SOX2 (Sox2), KLF4 (Klf4) and c-MYC (c-Myc) into immune cells. When using mature T or B cells as the source immune cells, it is preferable to introduce the 4 genes two times or more.

As described above, it has been difficult to induce iPS cells from normal mature T or B cells by introducing only the above 4 specific genes. Induction of iPS cells from these cells required lymphocytes with abnormal p53 or introduction of other genes. The inventors found that introduction of the 4 genes two or more times into normal mature T or B cells allows for induction of iPS cells from those lymphocytes without requiring introduction of additional genes (see Examples below).

In the second step, the iPS cells prepared in the first step are cocultured with OP9 cells, whereby the iPS cells are differentiated into immune cells. Examples of immune cells produced by the second step include T cells, B cells, NK cells, macrophages, and granulocytes.

There are no particular limitations to the method of differentiating iPS cells into immune cells by coculture with OP9 cells; it can be appropriately selected from any known method depending on the kind of immune cells to be differentiated. For example, coculture of iPS cells with typical OP9 cells results in differentiation into B cells, and coculture with Notch ligand delta like 1-expressing OP9 cells results in differentiation into T cells.

With this procedure it is possible to bulk produce desired immune cells semipermanently from source immune cells which are readily collected from the patient's body.

The immune cells produced by the production method according to an embodiment can be used for immunotherapy for individuals suffering from cancer, infectious disease or other disease. As the source of iPS cells, the production method uses immune cells which can be readily collected by taking a patient's blood sample without having to perform skin biopsy or other procedure. It is thus possible to reduce the patient's burden when applying the production method to immunotherapy.

### Examples

The present invention will be described in detail with reference to Examples, which however shall not be construed as limiting the scope of the invention thereto.

1. Generation of iPS Cells from B Cells

Using MACS beads (Miltenyi Biotech) CD19⁺ cells were isolated from the spleens of C57BL/6-Ly5.2 mice (RIKEN Bioresource center, Ibaraki, Japan) as peripheral B cells (purity: >98%). The isolated CD19⁺ cells were CD24⁺, CD45R(B220)⁺, and IgM⁺. The CD19⁺ cells were then activated by IL-4 and LPS. Specifically, the CD19⁺ cells were incubated for 24 hours in RPMI1640 medium supplemented with FCS (10% final conc.), penicillin (10 U/ml final conc.), streptomycin (100 μg/ml final conc.), glutamine (2 mM final conc.), sodium pyruvate (1 mM final conc.), and 2-mercaptoethanol (50 μM final conc.) in the presence of 10 ng/ml final conc. of IL-4 (Peprotech) and 1 μg/ml final conc. of LPS (Sigma-Aldrich).

Four reprogramming factors (Oct4, Sox2, Klf4, and c-Myc) were introduced into the activated CD19⁺ cells by retroviral transduction with centrifugation (780×g for 60 min), and then incubated in a 37° C., 5% CO₂ incubator. Four different pMXs vectors encoding Oct4, Sox2, Klf4 or c-Myc were used (see Non Patent Literature 3). Retroviruses were prepared in the same manner as reported previously (see Non patent Literatures 3 and 5), and 8 μg/ml final conc. of polybrene (Sigma-Aldrich) was added to the virus-containing supernatant. The viral transduction was done twice per two straight days.

Four days after the first transduction, the medium was replaced by iPS medium. Twelve days after the transduction, the cells were plated onto irradiated MEF feeder in ES medium in 100-mm dish, and 17 days after the transduction ES cell-like colonies were picked up. In the first experiment, ˜25 ES cell-like colonies were obtained from 4×10⁶ CD19⁺ cells. In the second experiment, ˜30 colonies were obtained from 1×10⁷ CD19⁺ cells.

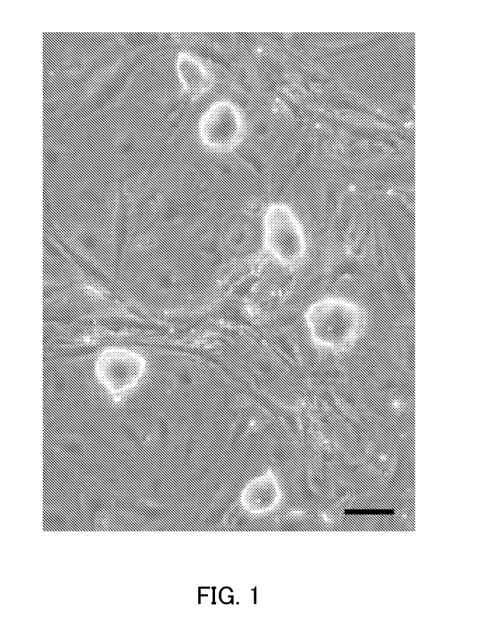

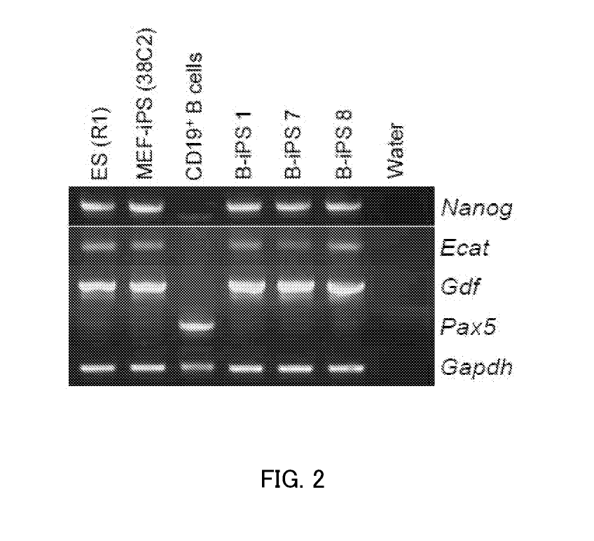

As shown in FIG. 1, B cell-derived iPS cells (B-iPS cells) were expandable and showed similar morphology to mouse ES cells and mouse embryonic fibroblast (MEF)-derived iPS cells (see Non Patent Literature 5). As shown in FIG. 2, the B-iPS cells expressed ES cell marker genes including Nanog, Ecat and Gdf as with ES cell line R1 and MEF-iPS cells, but did not express B-cell specific transcription factor, Pax5. In FIG. 2, B-iPS 1, B-iPS 7 and B-iPS 8 denote B-iPS cell lines prepared separately. The ES cell line R1 was generously obtained from Dr. Andras Nagy (Mount Sinai Hospital, Toronto, Canada). The MEF-iPS cells were purchased from RIKEN bioresource center (Ibaraki, Japan).

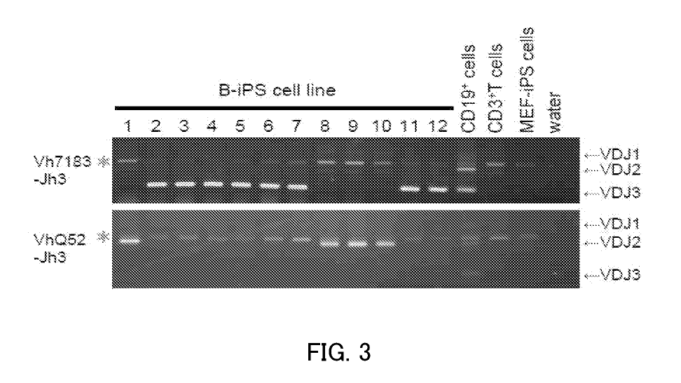

The B-iPS cells were investigated for the rearrangement of the B cell receptor (Bcr) genes. Specifically, genomic DNA was extracted from B-iPS cell line, splenic CD19⁺ cells, splenic CD3⁺ cells and MEF-iPS cells, and analyzed by genomic PCR for the occurrence of IgH V(D)J gene rearrangement (FIG. 3). Previously-reported PCR primers were used for the analysis of Bcr gene rearrangement (Ikawa T, Kawamoto H, Wright L Y et al., “Long-term cultured E2A-deficient hematopoietic progenitor cells are pluripotent”, Immunity, Vol. 20, pp. 349-360.; Kawamoto H, Ohmura K, Fujimoto S et al., “Extensive proliferation of T cell lineage-restricted progenitors in the thymus: an essential process for clonal expression of diverse T cell receptor beta chains” Eur. J. Immunol., Vol. 33, pp. 606-615.). In FIG. 3, the bands denoted by asterisks are non-specific bands. As shown in FIG. 3, 8 out of 12 separate B-iPS colonies showed VDJ3 band as splenic CD19⁺ cells did, whereas the other colonies showed VDJ2 band. These data indicate that the source of B-iPS cells was Bcr gene rearranged B cells, and that the rearranged Bcr gene was inherited to B-iPS cells.

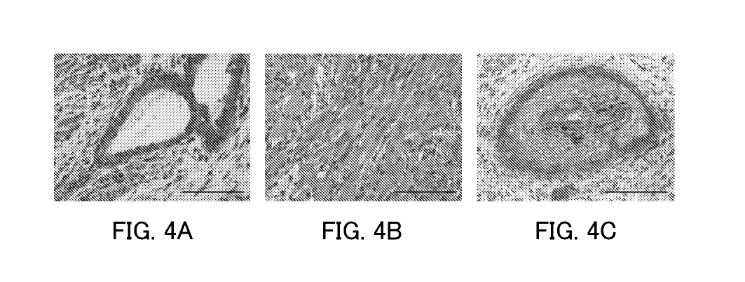

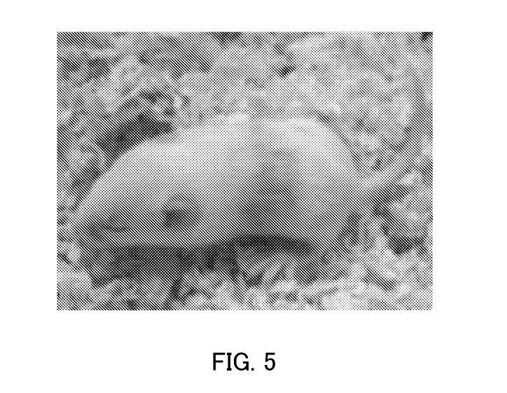

The B-iPS cells were examined for their ability to form teratoma. 1×10⁶ B-iPS cells suspended in PBS containing FCS (10% final conc.) were injected into the testis of NOD-SCID mice (Japan Clea, Tokyo). Four weeks after the injection, teratomas were visually observed in all of the injected mice, and the tumors were surgically dissected from the mice and fixed in 4% formaldehyde for histological observation, with the specimen stained with hematoxilin and eosin. As shown in FIGS. 4A to 4C, histological examination showed that the teratomas contained cell types representing all three embryonic germ layers. FIG. 4A shows ciliated cells (endoderm), FIG. 4B shows muscle fiber (mesoderm), and FIG. 4C shows dermal tissue (ectoderm). A controlled number of B-iPS cells was microinjected into ICR mouse blastocysts, which were then transferred to pseudopregnant female mice. As a result, it succeeded in generating chimeric mice with black and white hair from B-iPS cells, as shown in FIG. 5. These data indicate an establishment of iPS cells from mouse peripheral B cells by the Yamanaka 4 factors (see Non Patent Literature 3) without any additional factors.

2. T Lineage Differentiation from iPS Cells

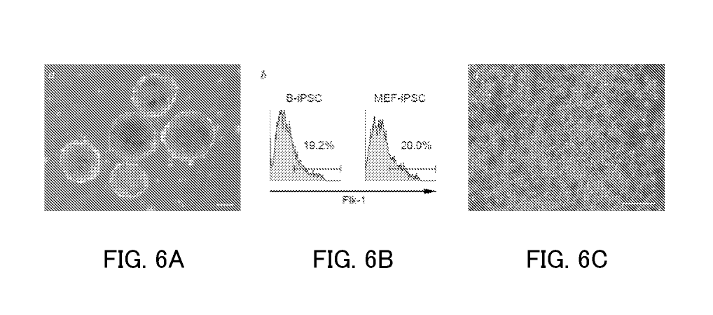

Differentiation of iPS cells (B-iPS cells or MEF-iPS cells) was started withdrawal of LIF from the culture in non-treatment dish. By day 5 of culture in LIF-free differentiation media, embryonic body-like spheres were formed from both B-iPS and MEF-iPS cells. FIG. 6A is a picture of B-iPS cell-derived spheres on day 5 of culture.

The generated embryonic body-like spheres contained mesoderm like cells which express Flk-1. FIG. 6B shows histograms indicating the patterns of Flk-1 expression in cells constituting the embryonic body-like spheres derived from iPS cells (B-iPS cells or MEF-iPS cells). Flow cytometry was done with a FACScalibur® instrument and analyzed by CellQuestPro® or FlowJo® software. Phycoerythrin-conjugated anti-Flk-1 antibody (clone 89B3A5; Biolegend, Tokyo) was used.

The Notch ligand delta like 1-expressing OP9 (OP9-DL1) cell lines were generous gift from Dr. Hiroshi Kawamoto (RCAI, RIKEN, Yokohama, Japan). The OP9-DL1 cells were cultured as monolayers in OP9 media, which is α-MEM supplemented with FCS (20% final conc.), 2-mercaptoethanol (0.1 mM final conc.), nonessential amino acids (0.1 mM final conc,), sodium pyruvate (1 mM final conc.), penicillin (10 U/ml final conc.), streptomycin (100 μg/ml final conc.), and sodium bicarbonate (2.2 g/liter final conc.).

The embryonic body-like spheres derived from iPS cells (B-iPS cells or MEF-iPS cells) were disrupted with 0.25% trypsin (Gibco-BRL). The resulting cell suspensions were plated on the monolayers of OP9-DL1 at a density of 6×10⁵ cells per 100-mm non-treated dish. The culture media contained F1t3 ligand (5 ng/ml final con.; R&D systems). On day 8 of culture, loosely adherent hematopoietic cells were harvested by gentle pipetting. Every 6 days thereafter, nonadherent iPS cell-derived hematopoietic cells were collected by vigorous pipetting, filtered through a 70-μm nylon mesh, and transferred onto OP9-DL1 monolayers in OP9 media. On day 8 of culture, another F1t3 ligand and exogenous IL-7 (5 ng/ml final conc.; R&D systems) were added. Both cytokines were added at all subsequent passages.

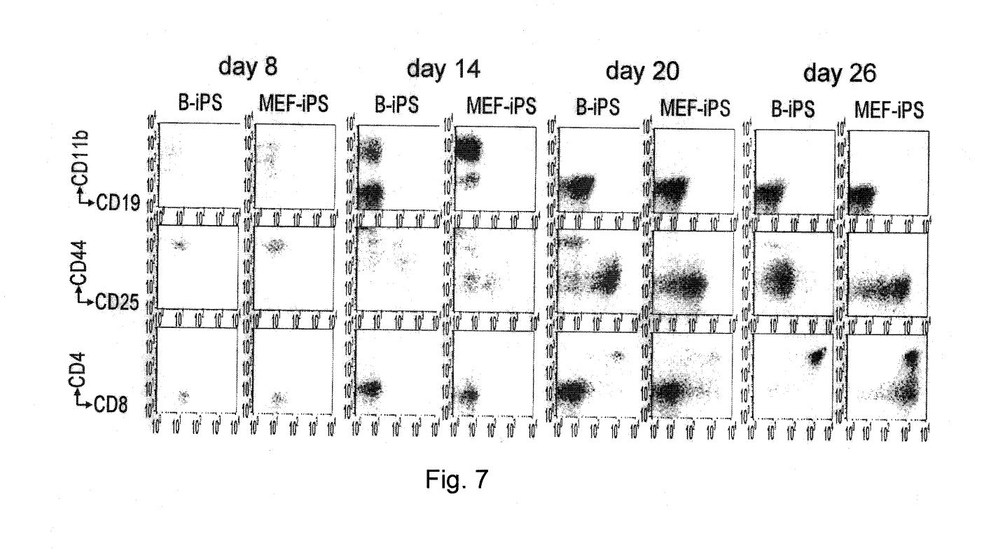

By day 14 of coculture with OP9-DL1 cells, the iPS cells (B-iPS cells or MEF-iPS cells) were transformed into lymphocyte-like cells. FIG. 6C is a picture of lymphocyte-like cells derived from B-iPS cells. Because these cells expressed CD25 and/or CD44 by day 14 of coculture as shown in FIG. 7, the iPS cells are considered to have been differentiated into T lineage in the same way that progenitor cells differentiate in the thymus. Phycoerythrin-conjugated anti-CD8 antibody (clone 53-6.7), anti-CD19 antibody (clone 1D3) and anti-CD25 antibody (clone 7D4), and allophycocyanin-conjugated anti-CD4 antibody (clone GK1.5), anti-CD11b antibody (clone M1/70) and anti-CD44 antibody (clone IM7) (all from Biolegend (Tokyo)) were used.

Rearrangement at the TCRβ locus (Tcrb) is a hallmark of T cell lineage commitment and is essential for the progression of CD4/CD8 double negative thymocytes to the double positive stage during normal αβ T cell development. To determine whether the T cells that develop from iPS cells cultured on OP9-DL1 cells undergo normal rearrangement of the TCRβ locus, the differentiated cells were stained at day 30 with various antibodies against TCRβ chain. Fluorescein isothiocyante-conjugated TCR panel (BD biosciences) was used.

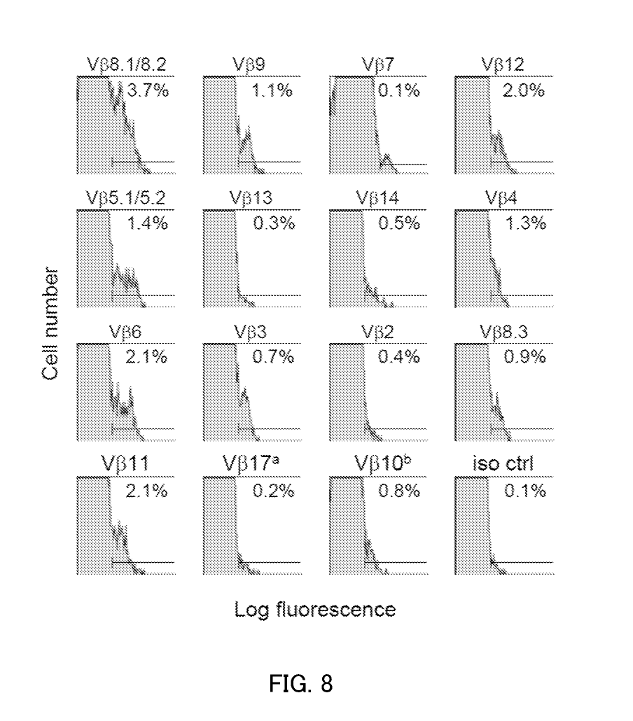

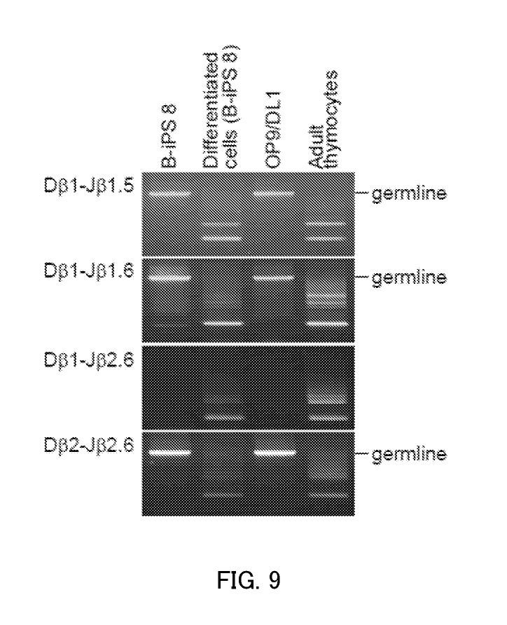

FIG. 8 shows histograms indicating the patterns of TcrVβ gene expression in B-iPS cell-derived T cells. MEF-iPS cell-derived T cells showed a similar pattern of TcrVβ gene expression. The diversity was also confirmed by genomic PCR. FIG. 9 is a gel electrophoresis picture showing the results of genomic PCR for B-iPS cells, differentiated cells from B-iPS cells, OP9-DL1 cells, and mouse adult thymocytes. Previously-reported PCR primers were used for the analysis of Tcr gene rearrangement (Ikawa T, Kawamoto H, Wright L Y et al., “Long-term cultured E2A-deficient hematopoietic progenitor cells are pluripotent”, Immunity, Vol. 20, pp. 349-360.; Kawamoto H, Ohmura K, Fujimoto S et al., “Extensive proliferation of T cell lineage-restricted progenitors in the thymus: an essential process for clonal expression of diverse T cell receptor beta chains” Eur. J. Immunol., Vol. 33, pp. 606-615.). The data shown in FIGS. 8 and 9 indicate that the iPS cell-derived T cells have the potential to generate a diverse TCR repertoire.

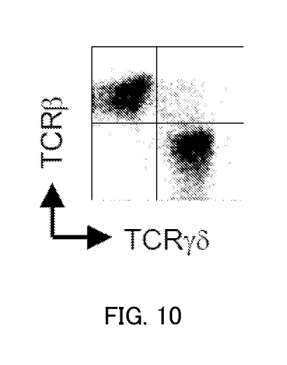

During normal thymocyte development, T cells bearing TCRαβ or TCRγδ develop in the thymus. To determine whether both populations of T cells develop from iPS cells cultured on OP9-DL1 cells, iPS-derived T cells were analyzed for surface expression of TCRαβ or TCRγδ. FIG. 10 shows a dot plot indicating the patterns of TCRβ and TCRγδ expressions in B-iPS cell-derived T cells. Allophycocyanin-conjugated anti-TCRβ antibody (clone H57-597), and phycoerythrin-conjugated anti-TCRγδ antibody (clone GL3) (both from Biolegend, Tokyo) were used. As shown in FIG. 10, it was demonstrated that both αβ T cells and γδ T cells were generated from B-iPS cells in this coculture system. Similarly, αβ T cells and γδ T cells were generated from MEF-iPS cells in this coculture system.

The iPS-derived T cells at day 20 and thereafter contained CD4/CD8 double positive cells and CD8 single positive cells (see FIG. 7). It was investigated whether the TCRs expressed on these T cells were indeed functional.

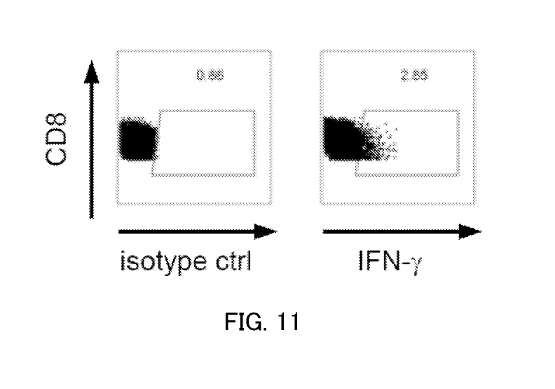

αβTCR^{hi}CD4⁻CD8⁺ T cells were sorted from the cultures at day 21, and 7.5×10⁴ T cells were stimulated for 3 days with plate-bound anti-CD3 antibody (10 μg/ml final conc.; clone 145-2C11) in the differentiation medium in the presence of IL-2 (1 ng/ml final conc.) and anti-CD28 antibody (1 μg/ml final conc.; clone 37.51). A further 6 hour-culture was done in the presence of PMA/Ionomycin. Intracellular staining for IFN-γ was done with Cytofix/Cytoperm® and GolgiStop® (BD Biosciences) according to the manufacturer's instructions. Phycoerythrin-conjugated anti-CD8 antibody (clone 53-6.7) and phycoerythrin-conjugated anti-IFN-γ antibody (clone XMG1.2) (both from Biolegend, Tokyo) were used. The stained cells were analyzed by flow cytometry. As a result, certain population of the iPS cell-derived T cells produced IFN-γ in response to the TCR stimulation, as shown in FIG. 11.

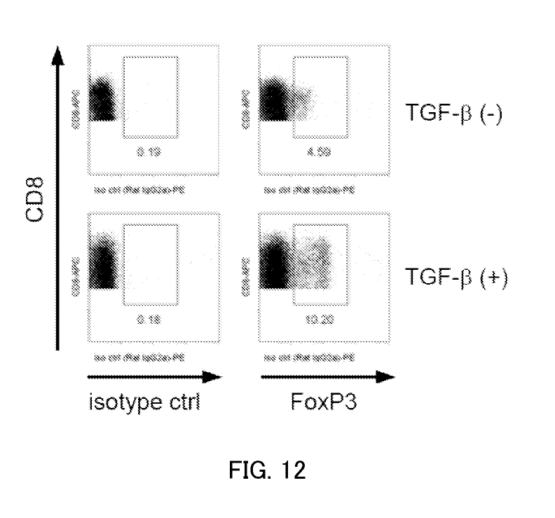

7.5×10⁴ isolated T cells were cultured for 2 days with plate-bound anti-CD3 antibody (10 μg/ml final conc.; clone 145-2C11) in differentiation medium in the presence of IL-2 (2 ng/ml final conc.) and TGF-β1 (5 ng/ml final conc.). As shown in FIG. 12, this enhanced the population of Foxp3-positive cells, which is the hallmark of regulatory T cells, as observed in naïve T cells derived from normal adult lymphoid tissue (Chen W, Jin W, Hardegen N et al., “Conversion of peripheral CD4⁺CD25⁻ naive T cells to CD4⁺CD25⁺ regulatory T cells by TGF-β induction of transcription factor Foxp3”, J. Exp. Med., Vol. 198, pp. 1875-1886.). These data indicate that the iPS cell-derived T cells generated in this coculture can respond to stimulation via TCR or cytokine receptors.

3. Analysis of Gene Expression in Differentiating iPS Cells

To elucidate the differentiation process of B-iPS cells at the molecular level, the expression of developmentally regulated genes was assessed by RT-PCR analysis. cDNA was generated with oligo dT primers and Superscript III (Invirtrogen) from total RNA samples. RT-PCR was performed with Amplitaq® (Applied biosystems) for ES markers and lymphocyte differentiation markers. Previously-reported primers were used (see Non Patent Literatures 3 and 17). PCR products were separated by agarose gel electrophoresis and visualized by ethidium bromide staining. All PCR products corresponded to expected molecular sizes.

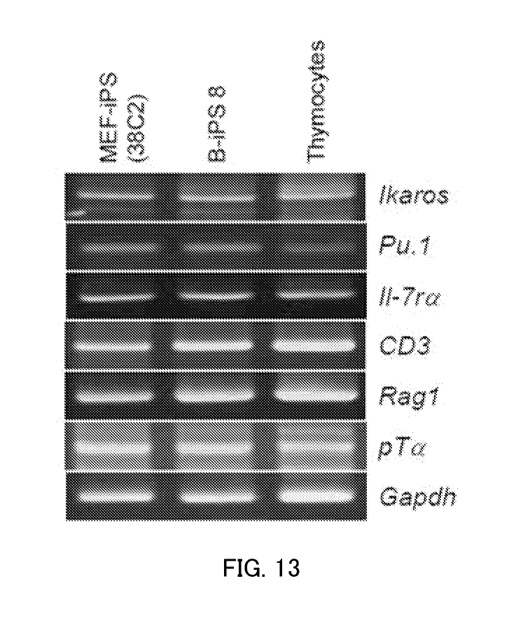

As seen in FIG. 13, a zinc finger transcription factor, Ikaros, and an Ets protein, PU.1, both of which are known to critically regulate hematopoiesis, showed significant expression in differentiating iPS cells cocultured with OP9-DL1. It was then investigated whether the gene encoding the interleukin 7 receptor (Il7r), which is required for the survival and proliferation of lymphocyte progenitors, was expressed; transcription of the Il7r gene was confirmed. Moreover, expression of CD3, Rag1 and pTα, which are essential for T lineage development, was observed as with normal thymocytes. These gene expressions are in agreement with the apparently normal development of T lineage from iPS cells in OP9-DL1 coculture (see FIGS. 7 to 12).

### INDUSTRIAL APPLICABILITY

The production method is useful for example as a cell preparation method in immunotherapy.

1. A production method of immune cells comprising:
generating induced pluripotent stem cells from source immune cells; and
differentiating the induced pluripotent stem cells into immune cells by coculture with OP9 cells.

2. The method according to claim 1, wherein the source immune cells are mature B cells, and the induced pluripotent stem cells are produced by introducing only reprogramming factors OCT4, SOX2, KLF4 and c-MYC into the mature B cells.

3. The method according to claim 2, wherein the induced pluripotent stem cells are produced by introducing the reprogramming factors two or more times into the mature B cells.

4. The method according to claim 1, wherein the OP9 cells are OP9-DL1 cells expressing Notch ligand delta like 1, and the induced pluripotent stem cells are differentiated into T cells by coculture with the OP9-DL1 cells.
